(19) United States
(12) Patent Application Publication
(10) Pub. No.: US 2022/0039671 A1
(43) Pub. Date: Feb. 10, 2022

(54) MULTI-TRANSMITTER SENSOR SYSTEM

(71) Applicant: Shifamed Holdings, LLC, Campbell, CA (US)

(72) Inventor: Brian J. Fahey, Menlo Park, CA (US)

(21) Appl. No.: 17/419,253

(22) PCT Filed: Dec. 31, 2019
(86) PCT No.: PCT/US2019/069106

§ 371 Date: Jun. 28, 2021

Related U.S. Application Data
(60) Provisional application No. 62/787,566, filed on Jan. 2, 2019.

(51) Int. Cl. A61B 5/0215; A61B 5/00

(52) U.S. Cl. CPC A61B 5/0215; A61B 2560/0214; A61B 5/0031

(57) ABSTRACT

Implantable relay devices that are configured to communicate with implantable sensing devices, the sensing devices spaced away from the implantable relay devices.

### INCORPORATION BY REFERENCE

This application claims priority to U.S. Provisional Application No. 62/787,566, filed Jan. 2, 2019, which is incorporated by reference herein for all purposes.

All publications and patent applications mentioned in this specification are herein incorporated by reference to the same extent as if each individual publication or patent application was specifically and individually indicated to be incorporated by reference.

The disclosures of the following references are incorporated by reference herein for all purposes: U.S. Pat. No. 8,593,107, 2008/0039904, 2010/0249888, 2013/0303942, U.S. Pat. No. 7,483,743, 2006/0122864. Relevant disclosures, descriptions, and examples in these references may be incorporated by reference into any suitable aspect of this disclosure, including without limitation any exemplary devices, systems, and methods of use.

### BACKGROUND

Invasive hemodynamic monitoring devices are typically delivered to a blood vessel, such as the pulmonary artery (PA), with the purpose of measuring blood pressure. These devices are primarily used for Heart Failure and left ventricular assist device (LVAD) patients to remotely monitor their internal blood pressure and to allow a care provider to clinically react to physiological changes that might occur.

One such device is CardioMEMS (Abbott, Ill.), which is a passive device (i.e., no internal power source) that is activated when excited by an external RF-antenna (the so-called wand). The pressure sensor then measures blood pressure and sends the information to an outside console (the hub or data-reader).

An active device (i.e., one that is powered, for example via a battery) may allow for several advantages over passive systems. For example, an active system may not require an external RF-antenna to excite a sensor circuit to acquire pressure data or transmit information to a data reader or hub. This may allow for measurement readings to be captured and transferred to a cloud-based or other central data repository with little to no active involvement from the patient, and may also enable on-demand measurements to be taken when a patient is not at-home or otherwise near the sensor activation equipment. It is expected that this routine would increase patient compliance, and have applicability to a wide range of implantable devices beyond those utilized for hemodynamic monitoring.

Many implanted sensors and devices are required to be small in size in order to access and reside in a targeted anatomical location without causing complications. Accordingly, for an implant to be an active system, the battery and electronics would generally also need to be small. Small batteries unfortunately have limited energy capacity, which could lead to unacceptably short device lifetimes and/or the requirement for more frequent recharging than is desirable.

Another limitation imposed by a small-sized implant is related to antenna size. Smaller antennae require higher transmission frequencies, which are more rapidly attenuated by mediums such as body tissues. Due to this signal attenuation, the initial transmission is generally required to be performed at high power to overcome the loss and ensure that a usable signal is detected by an outside reader. Larger antennae may transmit at lower frequencies that suffer less signal loss while traversing tissues but may not be of suitable size to be included on-board an implant device targeted for placement in the PA or other similarly-sized regions.

Without technological advances that enable a longer battery life, the advantages of an active implant may be outweighed by disadvantages related to device operating life, battery recharging frequency, and/or related matters. In addition, system designs that facilitate the process of recharging or replacing a battery would be useful and novel.

### SUMMARY

One aspect of the disclosure is an implantable relay device for communicating with an implantable sensing device, comprising: an anchoring portion with a collapsed delivery configuration and an expanded configuration that is sized and configured for secured anchoring within a subject's blood vessel; an energy storage device (e.g. battery) secured to the anchoring portion; and an implanted receiver in electrical communication with the battery, the receiver secured to the anchoring portion and configured to receive information from an implantable sensing device that is spaced away from the anchoring portion. The implantable relay device may be referred to herein as a secondary device.

Any of the implantable relay devices herein may include an anchoring portion that comprises a stent.

Any of the implantable relay devices herein may include an anchoring portion that has a cylindrical configuration in one or both of the collapsed configuration and the expanded configuration.

Any of the implantable relay devices herein may include an anchoring portion that comprises braided material.

Any of the implantable relay devices herein can include an anchoring portion that is a laser cut member.

Any of the implantable relay devices herein can include a receiver that comprises an antenna.

Any of the implantable relay devices herein can be configured to operate in a transmit mode and a receive mode.

Any of the implantable relay devices herein can include a receiver that is configured to receive signals emitted from a primary antenna on the implantable sensing device.

Any of the implantable relay devices herein can include at least one memory device, wherein the implantable relay device is configured to store in the memory information or data related to signals received from an implantable sensing device.

Any of the implantable relay devices herein can be adapted to transmit to an external device outside the subject data that is indicative of information or data received from the sensing device.

Any of the implantable relay devices herein can include a power storage device that is rechargeable.

Any of the implantable relay devices herein can include at least one of an acoustic transducer or an electromagnetic transmitter.

Any of the implantable relay devices herein can be configured to communicate charging signals to, and receive signals from, a sensing device that is positioned in a pulmonary artery when the implantable relay device is positioned in an inferior vena cava.

Any of the implantable relay devices herein can include an anchoring portion that includes a plurality of axially spaced anchoring sections, each of which is coupled to one or more adjacent anchoring sections by one or more connecting members, optionally wherein the anchoring sections can have a greater stiffness than the coupling members.

Any of the implantable relay devices herein can include a battery and a receiver, at least one of which can be disposed such that they do not prevent the anchoring portion from transitioning between expanded and collapsed configurations.

The disclosure also includes system that include any of the implantable relay devices herein and an implantable sensing device. An implantable sensing device can include one or more of a transmitter, optionally an antenna or a coil, a power source (e.g. a rechargeable battery), or a sensor

The disclosure also includes a method of positioning a pressure sensor and a relay device in a subject, comprising: positioning a pressure sensing device in a pulmonary artery, the pressure sensing device comprising a transmitter; and deploying an anchoring portion of an implantable relay device from a collapsed delivery configuration to an expanded configuration in a blood vessel of the subject such that the anchoring portion is spaced from the pressure sensing device, the implantable relay device comprising a battery and a receiver.

Positioning the pressure sensing device can include positioning the pressure sensing device in a right pulmonary artery. Deploying the anchoring portion can comprise deploying the anchoring portion in an inferior vena cava.

The disclosure also includes a method of positioning a pressure sensor and a relay device in a subject, comprising: positioning a sensing device in a first location in a vessel, the sensing device comprising a transmitter; positioning an anchoring portion including a battery in communication with the sensing device in a second location remote from the first location; and deploying the anchoring portion from a collapsed configuration to an expanded configuration.

### BRIEF DESCRIPTION OF THE FIGURES

FIG. 1A illustrates an exemplary secondary implant.

FIG. 1B illustrates an exemplary implantable system, including exemplary placement locations.

FIG. 2 illustrates an exemplary secondary implant with a plurality of axially spaced anchoring sections.

### DETAILED DESCRIPTION

The disclosure is related to implantable sensing devices (primary implanted devices) that are adapted and configured to be in communication with one or more secondary devices, which may be implantable. The secondary devices may communicate with the primary implanted devices (which includes the sensor) as part of a data transmission system. The secondary devices may be configured to be an implantable component, optionally that is adapted, configured, and sized for placement inside or nearby a vessel, inside or nearby another internal anatomical structure, or adapted, configured and sized for subcutaneous placement. The secondary devices serve as a communication relay between the primary implant devices and an external data reader hub.

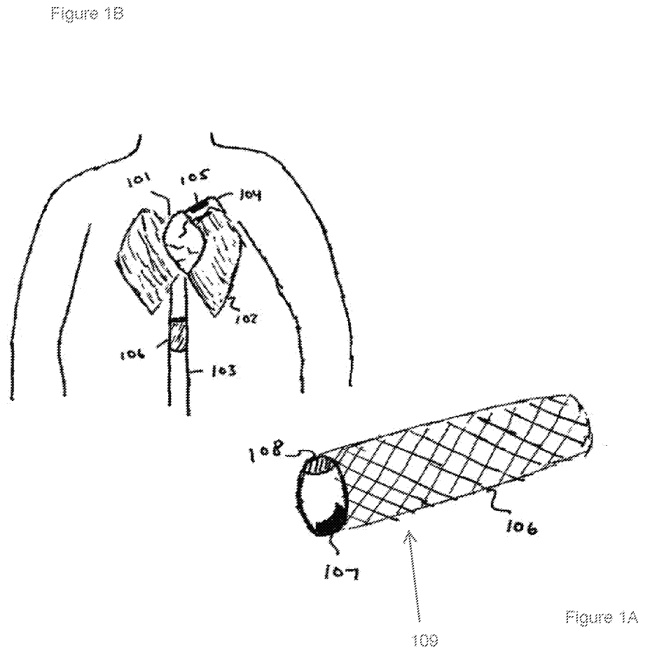

FIG. 1A illustrates an exemplary secondary implantable device 106. FIG. 1B illustrates an exemplary system after implantation, the system including secondary implantable device 106 and primary sensing device 105. In the exemplary embodiments shown in FIGS. 1A and 1B, secondary device 106 is implantable (at least temporarily) and includes an expandable and collapsible anchoring portion 109 that can include a stent or stent-like member (e.g., braided wires, laser cut tube, etc.) that is placed in an easily accessible vessel, for example (without limitation) in the inferior vena cava (“IVC”), superior vena cava (“SVC”), or a subclavian, femoral, or internal jugular vein. The secondary implant 106 also includes a secondary antenna 108 and a battery 107, both of which are secured to anchoring portion 109. The secondary implant may also contain one or more of an application specific integrated circuit (“ASIC”), a FPGA, or similar electronics that may contain some processing capabilities as well as limited on-board memory. In this embodiment, anchoring portion 109 has an expanded configuration (shown in FIGS. 1A and 1B) that is generally cylindrical. The configuration may vary, however, and may depend on the desired placement location. For example, the anchoring portion may have a pre-set hour-glass configuration when expanded.

Secondary implant 106 includes secondary antenna 108, which can be configured to operate both in transmit and receive modes. Secondary antenna 108 is adapted to receive signals emitted from a primary antenna on the primary implant 105, which in some embodiments can be implanted in the pulmonary artery and has a sensor to sense pulmonary arterial pressure. The primary antenna on primary implant 105 can be secured to a primary implant anchoring portion. The data corresponding to these received signals can be temporarily stored in a local memory that can be disposed on secondary implant 106. Secondary antenna 108 is also adapted to transmit these stored data (e.g., periodically, continuously) to an external data reader/hub, which may be located outside of the subject's body. Secondary implant 106 can further include one or more signal processing elements that can be configured to create a compressed output that is smaller than a full received data signal from the implantable sensing device 105. Secondary implant 106 can be configured to transmit the compressed output to a reader external to the body. Secondary implant 106 is an example of an implantable relay device, as that phrase is used herein.

FIG. 1B illustrates an exemplary system and exemplary placement locations. Heart 101, lung 102, IVC 103, and pulmonary artery 104 are shown. Primary implant 105, which can include a pressure sensor, is shown implanted in the pulmonary artery. Secondary implant 106 is shown implanted in the IVC, and is shown spaced away from primary implant 105. Secondary implant 106 and primary implant 105 are shown in different anatomical locations within the vasculature.

In some embodiments the anchoring portion 109 includes a stent or stent-like device, which can have a delivery, non-expanded configuration and a deployed, expanded configuration. Stents or stent-like devices are known. The non-expanded configuration can be sized to allow the anchoring portion 109 (and implant 106) to be advanced through the patient in a delivery device, such as a sheath or catheter. The anchoring portion of the secondary implant can be released and expanded once in a target location within the patient.

The anchoring portion (which might also be referred to herein as an anchoring member) can be manufactured using a variety of methods and techniques. For example, the anchoring portion can be formed from, for example without limitation, one or more elongate segments (e.g., wires) braided together, or a single member laser cut from a tubular member (e.g., nitinol tubular element). In some embodiments the anchoring portion has a length from 5 mm to 10 cm, for example.

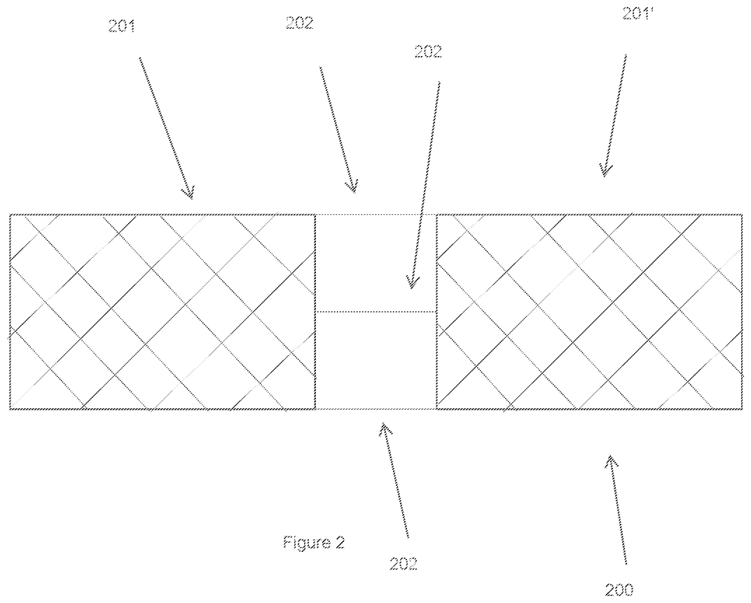

The anchoring portion may have a plurality of axially spaced stent sections that are axially coupled by one or more connecting members, where the connecting members may be the same or different material as the rest of the anchoring portion. For example the anchoring portion can include a plurality of the expandable member 109 that is shown in FIG. 1A, which can be strung together and coupled with one or more connecting members between adjacent stent or stent-like sections. FIG. 2 illustrates an exemplary secondary implant 200 that includes a plurality of axially spaced anchoring sections 201 and 201′, which may comprise any of the disclosure herein related to anchoring portions or anchoring members, including how non-anchoring components (e.g. antenna, battery, ASIC, memory) may be secured thereto. Anchoring sections 201 are shown coupled by a plurality of connectors 202 (three are shown in this example). The secondary implant may include more than two anchoring sections, such as three, four, five, six, seven or more. The secondary implant may include from 2 to 10 axially spaced anchoring sections, including any subrange included within that range (e.g., 4-7 sections). There may be advantages to having the anchoring portion separated into different axially spaced sections, but still all coupled together directly or indirectly. One or more electrical components (e.g., battery, receiver (e.g., antenna), application specific integrated circuit) can be coupled to one or more of the different regions of the anchoring member. One or more different anchoring sections may make it easier to expand and collapse if different electrical components are all coupled to the anchoring portion. In various embodiments, the system includes a plurality of anchoring sections, and the components of the system may be positioned in separate anchoring sections of the anchoring portion. The non-anchoring components may be wired or wirelessly electrically connected.

The one or more electrical components may be coupled to an inner surface or inner region of the anchoring portion (as shown in FIGS. 1A and 1B), but one or more electrical components may also be coupled to, or at least partially coupled to, an outer surface of the expandable member.

The one or more electrical components can be coupled to the anchoring member in a variety of ways. In some embodiments, portions of the anchoring member can move relative to adjacent section as it transitions from a delivery to expanded configuration. For example, elongated portions/arms of a stent or stent-like device may move relative to adjacent elongate portions/arm as it expands and collapses. The one or more electrical components, however, may be relatively stiff components that are not meant to flex or bend to any meaningful degree, or where the component is intended to have a desired configuration (e.g., a coiled antenna) when the anchoring member is expanded. The one or more electrical components can therefore be coupled to the anchoring member yet not impede the collapse and expansion of the anchoring member when in use.

In some embodiments, one or more electrical components may be secured to the anchoring member in a manner while still providing for a modest amount of relative movement between the anchoring member and one or more electrical components. For example, an electrical component like a battery could be sutured to a section of an anchoring member but with some slack to allow for some minor relative movement therebetween. If the anchoring member is placed in a vessel with blood flow, however, it may be undesirable to have too much relative movement between parts, as that could lead to friction and wear of one or more components. Additionally, one or more components could be disposed in a housing, where the housing is coupled to the anchoring member. The housing could be secured to the anchoring member in a way such as not to impeded expansion and collapse.

With any of the anchoring portions herein, elongate support posts (or similar components) can extend axially along at least a portion of the anchoring portion, to which one or more electrical components can be coupled. Additionally still, the anchoring member could include one or more flexible fabric materials that help secure one or more electrical components therein, wherein the flexible fabric material is secured to the structural support members (i.e., the structural member(s) of the anchoring member. In various embodiments, the anchoring member(s) can be covered with a polymer to provide one or more of promoting stabilization, improving biocompatibility, or maintaining long-term patency. In various embodiments, the anchoring member(s) include a fabric (e.g. polyester threads) to promote implantation and tissue ingrowth. In various embodiments, the anchoring system may instead, or in addition, include one or more expandable loops, for example nitinol loops deployed at the ends of the secondary implant, which can provide an outward pressure against the walls of a lumen when deployed into an expanded state from an initial collapsed state.

The exemplary system in FIGS. 1A and 1B as described herein provides a number of important advantages over previously-attempted systems. First, the system is designed to maximize the battery life on the primary implant (e.g., implant 105), which can have a relatively small, rechargeable battery therein. As the primary implant 105 no longer needs to transmit data over a relatively larger distance to a data reader outside of the body, and no longer needs to transmit signals that completely traverse high-loss body tissue media, the initial transmission power from the primary implant antenna may be markedly reduced. As such, the battery loss associated with each transmission will be minimized.

Secondly, the use of a secondary, larger, implant (e.g., implant 106) may allow for a larger battery (in terms of both size and capacity) and also a larger antenna to be used, and avoids having to place a larger battery and antenna in the relatively small primary implant. This larger antenna may thus be configured to transmit at lower frequencies, relative to a smaller antenna on the primary implant, enabling signals to leave the body with less attenuation. This allows transmissions to be initiated with lower output power, further extending the battery life of the secondary implant. In some preferred embodiments, the implantation location for the secondary implant is chosen to be an anatomical region that does not place excessive restrictions on the size of the battery or antenna. For example, in some preferred embodiments the second implant is positioned in a location within a larger diameter vessel, or in a subcutaneous pocket region proximate to the chest.

A third advantage of the exemplary system in FIGS. 1A and 1B involves improvements in a battery recharging paradigm. Traditionally, the recharging of a battery on an implanted device may represent a cumbersome process. Non-invasive charging methods (e.g., transcutaneous energy transmission) are often slow, inefficient, and/or imprecise and may require patient compliance with a recharging procedure. Invasive battery recharging/replacement procedures may allow for faster replenishment of battery power, but may be compromised by other disadvantages. For example, invasive recharging of a device implanted in the pulmonary artery may require a catheterization traversing the chambers of the right heart, leading to inherent time, cost, and patient-risk disadvantages that may make it non-ideal in some situations. Using the system described in this disclosure, the principal battery recharging/replacement activities may involve the secondary implant, which by design will be in a more accessible location than the pulmonary artery. This can make recharging the secondary implant battery much easier than recharging a battery that is in an implant positioned in or proximate to the heart. For example, for a secondary implant positioned in a subclavian vein, the skill level and risk associated with placing a recharging catheter in the peripheral vasculature is substantially less than that associated with navigating through the heart to the pulmonary artery. Similarly, a physician (or other healthcare provider) needing to occasionally replace or recharge a battery on a subcutaneously positioned implant can represent a streamlined paradigm that is considered significantly more desirable than accessing the pulmonary artery, or assuming that patients will be compliant with slow and/or cumbersome at-home recharging methods.

Any of the sensing devices herein, including those in the Claims section, can include one or more sensors, any one of which can be a pressure sensor. Any of sensors herein can be, for example without limitation, a piezoelectric, MEMS, acoustic, or fluid column sensor.

One or both of the secondary implant (relay device) and the primary implant (sensing device) can be an active device (i.e., one that is powered, for example via a battery), that can provide for several advantages over passive systems. For example, an active system may not require an external RF-antenna to excite a sensor circuit to acquire pressure data or transmit information to a data reader or hub. This may allow for measurement readings to be captured and transferred to a cloud-based or other central data repository with little to no active involvement from the patient, and may also enable on-demand measurements to be taken when a patient is not at-home or otherwise near the sensor activation equipment. It is expected that this routine would increase patient compliance, and have applicability to a wide range of implantable devices beyond those utilized for hemodynamic monitoring.

Any of the secondary devices (relay devices) herein (including in any Claims) that include a battery do not necessarily need to have a battery as the power or energy source. The relay devices herein can have other sources of energy that are not batteries. The secondary devices herein are thus understood to include one or more onboard energy capture elements and one or more energy storage components that are configured to store power for periods greater that a few seconds or minutes.

For example, any of the secondary device herein can include one or more super capacitor or other energy storage devices that are not batteries.

Any of the secondary implants (relay devices) herein, including those set forth in the claims, can include one or more antennas. For example, a first antenna can be configured to transmitting information to an external device (optionally also receiving information from the sensing device), and a second antenna can be configured for receiving energy to, for example, recharging a battery.

Any of the disclosure described in U.S. Pat. No. 8,593,107, US 2008/0039904, US 2010/0249888, US 2013/0303942, U.S. Pat. No. 7,483,743, and US 2006/0122864 related to descriptions of electrical coupling and communication of various electrical components is incorporated by reference herein for all purposes and can be integrated into any aspect of the disclosure herein, including devices, system, and methods.

1. An implantable relay device for communicating with an implantable sensing device, comprising:
an anchoring portion with a collapsed delivery configuration and an expanded configuration that is sized and configured for secured anchoring within a subject's blood vessel;
an energy storage device secured to the anchoring portion; and
an implanted receiver in electrical communication with the battery, the receiver secured to the anchoring portion and configured to receive information from an implantable sensing device that is spaced away from the anchoring portion.

2. The implantable relay device of claim 1, wherein the anchoring portion comprises a stent.

3. The implantable relay device of claim 1, where the anchoring portion has a cylindrical configuration in one or both of the collapsed configuration and the expanded configuration.

4. The implantable relay device of claim 1, wherein the anchoring portion comprises braided material.

5. The implantable relay device of claim 1, where the anchoring portion is a laser cut member.

6. The implantable relay device of claim 1, wherein the receiver comprises an antenna.

7. The implantable relay device of claim 1, wherein the receiver is configured to operate in a transmit mode and a receive mode.

8. The implantable relay device of claim 1, wherein the receiver is configured to receive signals emitted from a primary antenna on the implantable sensing device.

9. The implantable relay device of claim 1, further comprising at least one memory device, wherein the implantable relay device is configured to store in the memory information or data related to signals received from the implantable sensing device.

10. The implantable relay device of claim 1, further adapted to transmit to an external device outside the subject data that is indicative of information or data received from the sensing device.

11. The implantable relay device of claim 1, wherein the battery is rechargeable.

12. The implantable relay device of claim 1, wherein the relay device comprises at least one of an acoustic transducer or an electromagnetic transmitter.

13. The implantable relay device of claim 1, wherein the device is configured to communicate charging signals to, and receive signals from, the sensing device that is positioned in a pulmonary artery when the implantable relay device is positioned in an inferior vena cava.

14. The implantable relay device of claim 1, wherein the anchoring portion includes a plurality of axially spaced stent (or stent-like) portions, each of which is coupled to one or more adjacent stent portions by one or more connecting members, optionally wherein the stent portions can have a greater stiffness than the coupling members.

15. The implantable relay device of claim 1, wherein at least one of the battery and the receiver are disposed such that they do not prevent the anchoring portion from transitioning between the expanded and the collapsed configurations.

16. The implantable relay device of claim 1, wherein at least one of the battery and the receiver are coupled to the anchoring portion in a manner that allows for relative movement with the anchoring portion.

17. The implantable relay device of claim 1, further comprising a housing in which at least one of the battery and the receiver are disposed.

18. The implantable relay device of claim 1, further comprising a housing in which at least one electrical component is disposed, optionally wherein the at least one electrical component can be at least one of the battery and the receiver.

19. The implantable relay device of claim 1, further comprising at least one fabric member coupled to the anchoring portion, and optionally wherein the fabric member at least partially houses therein at least one electrical component.

20. The implantable relay device of claim 1, further comprising one or more signal processing elements configured to create a compressed output that is smaller than a full received data signal from the implantable sensing device, the implantable relay device configured to transmit the compressed output to a reader external to the body.

21. A system that includes any of the implantable relay devices of claim 1, further comprising the implantable sensing device.

22. The system of claim 21, wherein the implantable sensing device comprises a transmitter.

23-24. (canceled)

25. A method of positioning a pressure sensor and a relay device in a subject, comprising:
positioning a pressure sensing device in a pulmonary artery, the pressure sensing device comprising a transmitter; and
deploying an anchoring portion of an implantable relay device from a collapsed delivery configuration to an expanded configuration in a blood vessel of the subject such that the anchoring portion is spaced from the pressure sensing device, the implantable relay device comprising a battery and a receiver.

26. The method of claim 25, wherein positioning the pressure sensing device comprises positioning the pressure sensing device in a right pulmonary artery.

27. The method of claim 25, wherein deploying the anchoring portion comprises deploying the anchoring portion in an inferior vena cava.

28. The method of claim 25, wherein the anchoring portion is a stent or stent-like device, and optionally wherein the expanded configuration is cylindrical.

29. The method of claim 25, wherein the deploying step comprises positioning the anchoring portion radially outside of the battery and the receiver when the anchoring portion is in the expanded configuration.

30. The method of claim 25, wherein the pressure sensing device and the implantable relay device are in wired electrical communication.

31. The method of claim 25, further comprising recharging the battery in the implantable relay device, optionally with a catheter-based recharging device.

32. The method of claim 25, wherein the implantable relay device provides power to the pressure sensing device, the pressure sensing device optionally including a rechargeable battery.

33. The method of claim 25, wherein the implantable relay device receives information from the pressure sensing device that is indicative of sensed blood pressure.

34. The method of claim 25, further comprising transmitting information from the implantable relay device, optionally a compressed output, to an external device positioned outside the subject.

35. A method of positioning a pressure sensor and a relay device in a subject, comprising:
positioning a sensing device in a first location in a vessel, the sensing device comprising a transmitter;
positioning an anchoring portion including a battery in communication with the sensing device in a second location remote from the first location; and
deploying the anchoring portion from a collapsed configuration to an expanded configuration.

36. The method of claim 35, wherein the anchoring portion includes an implantable relay device.

37. The method of claim 35, wherein the anchoring portion includes a receiver, which is optionally configured to operate in a receive mode and a transmit mode.

38. The method of claim 35, wherein the second location is in the same vessel as the first location.

39. The method of claim 35, wherein the first location is in a pulmonary artery.

40. The method of claim 35, wherein the sensing device comprises an expandable anchor, the method further comprising deploying the sensing device from a collapsed configuration to an expanded configuration.
